(19) United States
(12) Patent Application Publication
(10) Pub. No.: US 2004/0242943 A1
(43) Pub. Date: Dec. 2, 2004

(54) Process for the production of fluoroethane and use of the produced fluoroethane

(76) Inventors: Kazunari Kaga, Kanagawa (JP); Hiromoto Ohno, Kanagawa (JP); Toshio Ohi, Tokyo (JP)

Correspondence Address: SUGHRUE MION, PLLC, 2100 PENNSYLVANIA AVENUE, N.W., SUITE 800, WASHINGTON, DC, 20037, US

(21) Appl. No.: 10/477,322

(22) PCT Filed: Mar. 7, 2003
(86) PCT No.: PCT/JP03/02728

Related U.S. Application Data
(60) Provisional application No. 60/364,035, filed on Mar. 15, 2002.

(30) Foreign Application Priority Data
Mar. 11, 2002 (JP) 2002-064830

(51) Int. Cl. C07C 19/08

(52) U.S. Cl. 570/123

(57) ABSTRACT

A high-purity pentafluoroethane is produced through a process comprising (1) a step of fluorinating tetrachloroethylene to obtain a crude pentafluoroethane containing impurities and (2) a step of bringing the crude pentafluoroethane containing impurities into contact with oxygen and/or an oxygen-containing compound in the presence of a catalyst.

### CROSS-REFERENCE TO RELATED APPLICATION

This application is an application filed under 35 U.S.C. § 111(a) claiming benefit, pursuant to 35 U.S.C. § 119(e)(1), of the filing date of the Provisional Application 60/364,035 filed Mar. 15, 2002, pursuant to 35 U.S.C. § 111(b).

### TECHNICAL FIELD

The present invention relates to a process for the production of pentafluoroethane, a process for producing hexafluoroethane using pentafluoroethane obtained by the production process, and use of the obtained pentafluoroethane.

### BACKGROUND ART

Pentafluoroethane (CF₃CHF₂) is used as a low-temperature refrigerant or as an etching gas and is also used as a starting material for the production of hexafluoroethane (CF₃CF₃).

As for the production of pentafluoroethane, various methods have been heretofore known.

For example, these are:

(1) a method of fluorinating tetrachloroethylene (CCl₂═CCl₂) or a fluorinated product thereof with hydrogen fluoride (see, Japanese Unexamined Patent Publication No. 8-268932 and Japanese International Application Domestic Publication No. 9-511515),

(2) a method of subjecting chloropentafluoroethane (CF₃CF₂Cl) to hydrogenolysis (see, Japanese Patent No. 2,540,409), and

(3) a method of reacting a fluorine gas with a halogen-containing ethylene (see, Japanese Unexamined Patent Publication No. 1-38034).

When these production methods are used, various impurities such as chlorofluorocarbons (CFC), hydrochlorofluorocarbons (HCFC) and hydrofluorocarbons (HFC) are contained in pentafluoroethane which is the objective substance.

In order to obtain high-purity pentafluoroethane, these impurities must be removed as much as possible. Among these impurities, various purification methods have been proposed to remove chlorofluorocarbons not only for the purpose of achieving high purity but also for preventing the depletion of the ozone layer. In particular, chloropentafluoroethane is close to pentafluoroethane in its boiling point and difficult to separate by normal distillation and, therefore, various purification methods have been proposed.

For example, these are:

(1) a method by extractive distillation (see, Japanese International Application Domestic Publication No. 9-508626)

(2) a method of subjecting chloropentafluoroethane to hydrogenolysis (see, Japanese Unexamined Patent Publication No. 8-301801), and

(3) a method of removing chloropentafluoroethane after fluorinating it with hydrogen fluoride (HF) (see, Japanese Unexamined Patent Publication No. 2001-48816).

On the other hand, as for the purification method for separating impurities comprising hydrochlorofluorocarbons or hyrofluorocarbons, only a few methods have been proposed. For example, Japanese International Application Domestic Publication No. 9-508627 describes a purification method by extractive distillation. Among hydrochlorofluorocarbons and hydrofluorocarbons, difluoromethane (CH₂F₂) and 1,1,1-trifluoroethane (CF₃CH₃) are known to form an azeotropic mixture with pentafluoroethane and these compounds are very difficult to separate from pentafluoroethane.

When pentafluoroethane is produced by a method containing hydrogenolysis, 1,1,1-trifluoroethane is very often produced as a by-product due to an excess hydrogenation-dehalogenation reaction and this compound is contained in pentafluoroethane in a relatively large amount. For removing 1,1,1-trifluoroethane contained in pentafluoroethane, a method by extractive distillation is known. However, this method has a problem that a plurality of expensive facilities, such as distillation towers, are necessary and the equipment cost is very high.

### DISCLOSURE OF INVENTION

Under these circumstances, an object of the present invention is to provide an industrially advantageous process for producing high-purity pentafluoroethane which can be used as a low-temperature refrigerant or an etching gas or as a starting material for the production of high-purity hexafluoroethane; a process for producing hexafluoroethane using pentafluoroethane produced by the above-described process; and uses of the obtained pentafluoroethane.

As a result of extensive investigations to solve the above-mentioned problems, the present inventors have found that those problems can be solved by a process for producing pentafluoroethane, comprising (1) a step of fluorinating tetrachloroethylene to obtain a crude pentafluoroethane containing impurities and (2) a step of bringing the crude pentafluoroethane containing impurities into contact with oxygen and/or an oxygen-containing compound in the presence of a catalyst. The present invention has been accomplished based on this finding.

The production process of pentafluoroethane of the present invention comprises the following steps:

(1) a step of fluorinating tetrachloroethylene to obtain a crude pentafluoroethane, and

(2) a step of bringing the crude pentafluoroethane into contact with oxygen and/or an oxygen-containing compound in the presence of a catalyst.

The crude pentafluoroethane used in the step (2) is preferably obtained through a further step of being contacted with hydrogen.

The temperature in the step (2) is preferably from 150 to 400° C.

The catalyst is preferably a supported or bulk catalyst mainly comprising trivalent chromium oxide.

The catalyst is also preferably a supported catalyst mainly comprising at least one metal selected from the group consisting of palladium, rhodium, ruthenium, rhenium, platinum and gold.

The support for use in the supported catalyst is preferably alumina, fluorinated alumina or zeolite.

The crude pentafluoroethane may contain as impurities at least one compound selected from the group consisting of fluoromethane, difluoromethane, fluoroethane, 1,1-difluoroethane, 1,2-difluoroethane, 1,1,1-trifluoroethane and 1,1,2-trifluoroethane.

The total amount of impurities contained in the crude pentafluoroethane is 2 vol % or less.

In another embodiment, the production process of pentafluoroethane of the present invention comprises bringing a crude pentafluoroethane into contact with oxygen and/or an oxygen-containing compound at 150 to 400° C. in the presence of a catalyst mainly comprising trivalent chromium oxide, and then separating impurities by distillation.

In still another embodiment, the production process of pentafluoroethane of the present invention comprises bringing a crude pentafluoroethane into contact with oxygen and/or an oxygen-containing compound at 150 to 400° C. in the presence of a supported catalyst mainly comprising at least one metal selected from the group consisting of palladium, rhodium, ruthenium, rhenium, platinum and gold, and then separating impurities by distillation.

The crude pentafluoroethane may contain at least trifluoroethane as impurities.

The concentration of oxygen and/or oxygen-containing compound is preferably from 0.1 to 20 vol %.

The present invention provides a pentafluoroethane having a total impurity amount of 500 vol ppm or less, which is obtained by any of these production processes.

The content of trifluoroethane contained as an impurity in pentafluoroethane is preferably 100 vol ppm or less.

The present invention also provides a refrigerant comprising the pentafluoroethane.

The production process of hexafluoroethane of the present invention comprises the following steps:

(1) a step of fluorinating tetrachloroethylene to obtain a crude pentafluoroethane,

(2) a step of bringing the crude pentafluoroethane into contact with oxygen and/or an oxygen-containing compound in the presence of a catalyst to obtain pentafluoroethane, and

(3) a step of reacting the pentafluoroethane obtained through the step (2) with a fluorine gas to obtain hexafluoroethane.

The crude pentafluoroethane used in the step (2) is preferably obtained through a further step of being contacted with hydrogen.

### BEST MODE FOR CARRYING OUT THE INVENTION

The present invention is described in detail below.

As described above, pentafluoroethane can be produced, for example, by a method of fluorinating tetrachloroethylene or a fluorination product thereof with hydrogen fluoride (HF) or a method of subjecting chloropentafluoroethane to hydrogenolysis. Whichever method is used for the production, the pentafluoroethane obtained through a general purification step such as distillation contains chloropentafluoroethane which is an impurity difficult to separate from pentafluoroethane. This chloropentafluoroethane must be separated so as to obtain high-purity pentafluoroethane, and also from the standpoint of preventing depletion of the ozone layer, it is required not to contain chloropentafluoroethane.

With respect to the method for separating chloro-pentafluoroethane contained in pentafluoroethane, for example, a method using hydrogenolysis, a method using extractive distillation and a method using adsorption have been proposed as described above. Among these methods, the method using hydrogenolysis can produce pentafluoroethane more inexpensively in view of equipment cost. One of the problems encountered, when a method containing a hyrogenolysis step is selected as the method for producing or purifying pentafluoroethane, is the production of difficultly separable hydrofluorocarbons (HFC) such as 1,1,1-trifluoroethane due to an excess hydrogenation reaction. In particular, difluoromethane and 1,1,1-trifluoroethane are difficult to separate by a general purification method because these substances are very close to pentafluoroethane in boiling points and also known to form an azeotropic mixture. For separating hydrofluorocarbons (HFC) contained in pentafluoroethane, a method by extractive distillation has been proposed but this method has a problem that a plurality of expensive facilities, such as distillation towers, are necessary and the equipment cost is very high.

The production process of pentafluoroethane of the present invention comprises (1) a step of fluorinating tetrachloroethylene to obtain a crude pentafluoroethane containing impurities, and (2) a step of bringing the crude pentafluoroethane into contact with oxygen and/or an oxygen-containing compound in the presence of a catalyst. The method for the step (1) is not particularly limited and, for example, tetrachloroethylene may be fluorinated through two steps using hydrogen fluoride (HF) in the presence of a catalyst to obtain a crude pentafluoroethane.

In the present invention, impurities contained in pentafluoroethane, such as hydrofluorocarbon (HFC), are brought into contact with oxygen and/or an oxygen-containing compound in the presence of a catalyst in the gas phase at a temperature elevated to 150 to 400° C., whereby any hydrofluorocarbon contained as an impurity is oxidized and converted to CO₂ or the like. For example, when difluoroethane or 1,1,1-trifluoroethane contained in pentafluoroethane is oxidized with oxygen, a reaction represented by the following formula (a) or (b) is considered to proceed:

CH₂F₂+O₂→CO₂+2HF formula (a)

CF₃CH₃+2O₂→2CO₂+3HF formula (b)

The main oxidation product is CO₂ and as a by-product, HF is produced.

The compound which is converted to CO₂ by this reaction includes fluoromethane, difluoromethane, fluoroethane, 1,1-difluoroethane, 1,2-difluoroethane, 1,1,1-trifluoroethane, 1,1,2-trifluoroethane and the like. In the case of using a production or purification method containing a hydrogenolysis step, the pentafluoroethane usually contains these compounds in a total amount of approximately thousands of vol ppm. These impurities must be removed to obtain high-purity pentafluoroethane.

In the production process of pentafluoroethane of the present invention, the total amount of impurities contained in the crude pentafluoroethane, such as hydrofluorocarbon (HFC), is preferably 2 vol % or less, more preferably 0.5 vol % or less, still more preferably 0.3 vol % or less. If the content of impurities such as hydrofluorocarbons exceeds 2 vol %, the reaction temperature must be high and the catalyst may have a short life-time.

The catalyst used for the reaction is preferably (i) a supported or bulk catalyst mainly comprising trivalent chromium oxide or (ii) a supported catalyst mainly comprising at least one metal selected from the group consisting of palladium, rhodium, ruthenium, rhenium, platinum and gold. Examples of the raw material which can be used include these metals, and oxides and salts of these metals. Examples of the support which can be used for the supported catalyst include alumina, fluorinated alumina and zeolite.

The catalyst (i) mainly comprising trivalent chromium oxide can be prepared, for example, by adding dropwise a basic substance such as ammonia in an aqueous solution of chromium metal salt to precipitate chromium hydroxide, washing/filtering/drying the precipitate, molding the obtained chromium hydroxide, and heat-treating the molded article in the presence of an inert gas such as nitrogen. The supported catalyst (ii) mainly comprising palladium, rhodium, ruthenium, rhenium, platinum and/or gold can be prepared, for example, by dissolving a salt of the metal in a water-soluble solvent such as water, methanol and acetone, immersing a support in the solution to adsorb necessary elements, distilling off the solvent and reducing the support with hydrogen under heat.

The temperature in the step (2) is preferably from 150 to 400° C., more preferably from 180 to 370° C. If the reaction temperature exceeds 400° C., the catalyst may have a short life-time and the number of kinds, and amount, of by-products not ascribable to the main reaction may increase.

The concentration of oxygen and/or oxygen-containing compound contained in the reaction substrate gas is preferably from 0.1 to 20 vol %. The oxygen may be high-purity oxygen or air, but a high-purity oxygen is preferred. If the oxygen concentration is less than 0.1 vol %, the conversion disadvantageously decreases due to insufficiency of oxygen necessary for the reaction, though this varies depending on the kind and amount of hydrofluorocarbons contained as an impurity in pentafluoroethane. On the other hand, if the oxygen concentration exceeds 20 vol %, an excess reaction proceeds to cause a decomposition reaction of pentafluoroethane which is the main component of the reaction substrate gas, and this is not preferred in view of profitability because the loss of pentafluoroethane increases. Examples of the oxygen-containing compound which can be used include nitrogen monoxide (NO), nitrous oxide (N₂O), nitrogen dioxide (NO₂) and ozone (O₃).

The production process of pentafluoroethane of the present invention can be performed under the above-described reaction conditions, however, if the reaction product contains CO₂, by-products not ascribable to the main reaction, such as hydrofluorocarbons, and acid contents such as HF, other than pentafluoroethane, and CO₂ and acid contents are preferably removed.

The acid contents may be removed, for example, by a method of bringing the reaction product into contact with a purifying agent or a method of bringing the reaction product into contact with water, an alkali aqueous solution or the like. The gas from which the acid contents are removed is preferably dehydrated using a dehydrating agent such as zeolite and then distilled to remove CO₂ and simultaneously those by-products not ascribable to the main reaction.

In another embodiment, the present invention provides a process for producing pentafluoroethane, comprising bringing a crude pentafluoroethane into contact with oxygen and/or an oxygen-containing compound at 150 to 400° C. in the presence of a catalyst mainly comprising trivalent chromium oxide, and then separating impurities by distillation.

In still another embodiment, the present invention provides a process for producing pentafluoroethane, comprising bringing a crude pentafluoroethane into contact with oxygen and/or an oxygen-containing compound at 150 to 400° C. in the presence of a supported catalyst mainly comprising at least one metal selected from the group consisting of palladium, rhodium, ruthenium, rhenium, platinum and gold, and then separating impurities by distillation.

The method for purification after the reaction is not particularly limited and the purification can be performed by commonly used distillation. With respect to the distillation method, for example, the following method may be used.

After the crude pentafluoroethane is contacted with oxygen and/or an oxygen-containing compound at 150 to 400° C. in a reactor, the resulting gas is introduced into a distillation tower. The inner pressure of the distillation tower is preferably from atmospheric pressure to 2 MPa. If the inner pressure is less than atmospheric pressure, a facility of reduced pressure system is disadvantageously necessary, whereas if it exceeds 2 MPa, a facility of high pressure system is necessary and this is not preferred. For example, in the case where the above-described catalytic reaction is performed using oxygen, a low boiling fraction containing oxygen is extracted from the top of the distillation tower and a high boiling fraction is extracted from the bottom of the distillation tower. At this time, the components extracted from the top and bottom sometimes contain pentafluoroethane which is the objective component. If this is so, respective components may be introduced into separate distillation towers and purified to recover the pentafluoroethane. When the component separated here is an intermediate for the production of pentafluoroethane, the component may be returned to the reaction step and re-used.

By such purification, pentafluoroethane having a higher purity can be obtained. The content of impurities contained is 500 vol ppm or less. The pentafluoroethane having a purity of 99.95 vol % or more can be analyzed by gas chromatography (GC) using TCD method or FID method, or gas chromatography-mass spectrometry (GC-MS).

Uses of the pentafluoroethane obtained by the production process of the present invention are described below.

The high-purity pentafluoroethane can be used as a substitute for chlorodifluoromethane (CHF₂Cl) which is a currently-used working fluid for low-temperature refrigerators, and also can be used as a raw material of mixed refrigerants which are other substitutes for chlorodifluoromethane, such as difluoromethane/pentafluoroethane/1,1,1,2-tetrafluoroethane and difluoromethane/pentafluoroethane.

Furthermore, the high-purity pentafluoroethane can be used as a starting material for the production of high-purity hexafluoroethane. Particularly, in the process of producing hexafluoroethane by a reaction of pentafluoroethane with a fluorine gas (F₂), when high-purity pentafluoroethane is used as a starting material, production of impurities which are difficult to separate from hexafluoroethane can be prevented, the latitude in setting the fluorination reaction conditions can be enlarged, the reaction can be stably controlled and the purification step can be simplified.

Accordingly, the present invention provides a process for producing hexafluoroethane, comprising (1) a step of fluorinating tetrachloroethylene to obtain a crude pentafluoroethane, (2) a step of bringing the crude pentafluoroethane into contact with oxygen and/or an oxygen-containing compound in the presence of a catalyst, and (3) a step of reacting the pentafluoroethane obtained through the step (2) with a fluorine gas.

The crude pentafluoroethane used in the step (2) is preferably obtained through a further step of being contacted with hydrogen.

The high-purity pentafluoroethane or a mixed gas thereof, with an inert gas such as He, N₂ and Ar, HCl, O₂, H₂ or the like, can be used as an etching gas in an etching step in the process of producing a semiconductor device. In the process of producing a semiconductor device such as an LSI, a TFT and an organic EL device, a thin or thick film is formed using a CVD method, a sputtering method or a vapor deposition method and a circuit pattern is formed by etching, where a mixed gas containing the pentafluoroethane can be used as an etching gas. The etching using pentafluoroethane can be performed under various dry etching conditions such as plasma etching and microwave etching.

The present invention is described in greater detail below, however, the present invention is not limited to these Examples.

### RAW MATERIAL EXAMPLE OF PENTAFLUOROETHANE

Tetrachloroethylene and hydrogen fluoride were introduced into a first reactor filled with a catalyst to produce a gas mainly comprising 1,1,1-trifluoro-2,2-dichloroethane and 1,1,1,2-tetrafluoro-2-chloroethane which are intermediates. This gas was introduced together with HF into a second reactor to produce pentafluoroethane. The produced pentafluoroethane was distilled to obtain pentafluoroethane containing 0.5% of chloropentafluoroethane as an impurity.

This pentafluoroethane was reacted with hydrogen in the presence of a commercially available hydrogenation catalyst (reaction pressure: 0.35 MPa, reactor temperature: 280° C., H₂/chloropentafluoroethane molar ratio=5). The acid content was removed from the resulting mixed gas by a known method and the residue was purified by distillation, as a result, a distillate mainly comprising pentafluoroethane was obtained. This distillate was analyzed by gas chromatography and found to be a mixed gas having the composition shown in Table 1.

TABLE 1

| Components | Concentration (vol %) |
|---|---|
| CF₃CHF₂ | 99.7171 |
| CF₃CF₂Cl | 0.0005 |
| CF₃CH₂F | 0.0201 |
| CF₃CH₃ | 0.2621 |
| CHF₃ | 0.0002 |

### PRODUCTION EXAMPLE OF CATALYST (CATALYST 1)

Chromium nitrate nonahydrate was dissolved in water and mixed with 28 wt % of aqueous ammonia while stirring to obtain a chromium hydroxide slurry. This was separated by filtration, thoroughly washed with water and then dried at 120° C. The obtained lump was pulverized, mixed with graphite and pelletized by a tablet molding machine. The obtained pellet was baked at 400° C. for 4 hours in a N₂ stream to obtain Catalyst 1 mainly comprising trivalent chromium oxide.

### PRODUCTION EXAMPLE OF CATALYST (CATALYST 2)

Chloroplatinic acid was dissolved in water and a 3 mmφ spherical alumina support was dipped in the resulting solution and adsorbed the platinum salt. Thereafter, the solvent was distilled off at a temperature of 100° C. and the residue was baked in an air at 300° C. and then hydrogen-reduced at 350° C. The percentage of platinum supported in the obtained Platinum Catalyst 2 was 0.25%.

### EXAMPLE 1

A catalyst (Catalyst 1) (100 ml) was filled in an Inconel 600-made reactor having an inner diameter of 1 inch and a length of 1 m and kept at a temperature of 300° C. while passing a nitrogen gas. Subsequently, oxygen was supplied at a flow rate of 2.0 NL/hr, a gas having the composition shown in Table 1 was supplied at a flow rate of 38.0 NL/hr, the supply of nitrogen gas was then stopped and the reaction was started. After 2 hours, the outlet gas from the reactor was washed with an aqueous potassium hydroxide solution to remove the acid content, then contacted with Molecular Sieves 3A (produced by Union Showa K.K.) and dried. The resulting dried gas mainly comprising pentafluoroethane was collected under cooling and purified by distillation. The gas after the purification was analyzed by gas chromatography and found to be a gas having the composition shown in Table 2.

TABLE 2

| Components | Concentration [vol %] |
|---|---|
| CF₃CHF₂ | 99.9665 |
| CF₃CF₂Cl | 0.0004 |
| CF₃CH₂F | 0.0126 |
| CF₃CH₃ | 0.0204 |
| CHF₃ | 0.0001 |

### EXAMPLE 2

Pentafluoroethane was obtained by the same operation as in Example 1 except for using Catalyst 2. The gas after the purification was analyzed and found to have the composition shown in Table 3.

TABLE 3

| Components | Concentration [vol %] |
|---|---|
| CF₃CHF₂ | 99.9840 |
| CF₃CF₂Cl | 0.0004 |
| CF₃CH₂F | 0.0101 |
| CF₃CH₃ | 0.0054 |
| CHF₃ | 0.0001 |

### EXAMPLE 3

A nitrogen gas was supplied to a nickel-made reactor having an inner diameter of 1 inch and a length of 50 cm (employing a heating system using an electric heater; the reactor had been subjected to a passivation treatment with a fluorine gas at a temperature of 500° C.) through two gas inlets at a total flow rate of 30 NL/hr and the reactor was kept at a temperature of 420° C. Subsequently, HF was passed through the above-described two gas inlets at a total flow rate of 50 NL/hr and the mixed gas mainly comprising pentafluoroethane obtained in Example 1 was introduced through one gas inlet at a flow rate of 3.5 NL/hr. Also, a fluorine gas was introduced through another gas inlet at a flow rate of 3.85 NL/hr, thereby performing a reaction. After 3 hours, the outlet gas from the reactor was contacted with an aqueous potassium hydroxide solution and an aqueous potassium iodide solution to remove HF and unreacted fluorine gas. Thereafter, the gas was contacted with a dehydrating agent and thereby dried and the dried gas was collected under cooling and then purified by distillation. The gas after the purification was analyzed by the TCD method, the FID method and the ECD method of gas chromatography and the GC-MS method. The results are shown in Table 4.

TABLE 4

| Components | Concentration [vol %] |
|---|---|
| CF₃CF₃ | >99.9998% |
| CF₄ | <0.4 vol ppm |
| CF₃CF₂Cl | <0.1 vol ppm |
| CF₃CHF₂ | <0.5 vol ppm |
| SF₆ | <0.4 vol ppm |

As is apparent from the analysis results shown in Table 4, the hexafluoroethane contained almost no other impurities and a high-purity hexafluoroethane was obtained.

As described in the foregoing pages, in accordance with the present invention, high-purity pentafluoroethane can be obtained. The pentafluoroethane obtained by the present invention can be used as a low-temperature refrigerant, an etching gas or a starting material for the production of high-purity hexafluoroethane.

1. A process for producing pentafluoroethane, comprising the following steps:
(1) a step of fluorinating tetrachloroethylene to obtain a crude pentafluoroethane, and
(2) a step of bringing said crude pentafluoroethane into contact with oxygen and/or an oxygen-containing compound in the presence of a catalyst.

2. The process as claimed in claim 1, wherein the crude pentafluoroethane used in the step (2) is obtained through a further step of being contacted with hydrogen.

3. The process as claimed in claim 1 or 2, wherein the temperature in the step (2) is from 150 to 400° C.

4. The process as claimed in any one of claims 1 to 3, wherein the catalyst is a supported or bulk catalyst mainly comprising trivalent chromium oxide.

5. The process as claimed in any one of claims 1 to 3, wherein the catalyst is a supported catalyst mainly comprising at least one metal selected from the group consisting of palladium, rhodium, ruthenium, rhenium, platinum and gold.

6. The process as claimed in claim 4 or 5, wherein the support for use in the supported catalyst is alumina, fluorinated alumina or zeolite.

7. The process as claimed in any one of claims 1 to 6, wherein said crude pentafluoroethane comprises at least one compound selected from the group consisting of fluoromethane, difluoromethane, fluoroethane, 1,1-difluoroethane, 1,2-difluoroethane, 1,1,1-trifluoroethane and 1,1,2-trifluoroethane as impurities.

8. The process as claimed in any one of claims 1 to 7, wherein the total amount of impurities contained in said crude pentafluoroethane is 2 vol % or less.

9. A process for producing pentafluoroethane, comprising bringing a crude pentafluoroethane into contact with oxygen and/or an oxygen-containing compound at 150 to 400° C. in the presence of a catalyst mainly comprising trivalent chromium oxide, and then separating impurities by distillation.

10. A process for producing pentafluoroethane, comprising bringing a crude pentafluoroethane into contact with oxygen and/or an oxygen-containing compound at 150 to 400° C. in the presence of a supported catalyst mainly comprising at least one metal selected from the group consisting of palladium, rhodium, ruthenium, rhenium, platinum and gold, and then separating impurities by distillation.

11. The process as claimed in claim 9 or 10, wherein said crude pentafluoroethane comprises at least trifluoroethane as impurities.

12. The process as claimed in any one of claims 9 to 11, wherein the concentration of oxygen and/or oxygen-containing compound is from 0.1 to 20 vol %.

13. A pentafluoroethane having a total impurity amount of 500 vol ppm or less, which is obtained by the process claimed in any one of claims 1 to 12.

14. The pentafluoroethane as claimed in claim 13, wherein the content of trifluoroethane contained as an impurity is 100 vol ppm or less.

15. A refrigerant comprising the pentafluoroethane claimed in claim 13 or 14.

16. A process for producing hexafluoroethane, comprising the following steps:
(1) a step of fluorinating tetrachloroethylene to obtain a crude pentafluoroethane,
(2) a step of bringing said crude pentafluoroethane into contact with oxygen and/or an oxygen-containing compound in the presence of a catalyst to obtain pentafluoroethane, and
(3) a step of reacting the pentafluoroethane obtained through the step (2) with a fluorine gas to obtain hexafluoroethane.

17. The process as claimed in claim 16, wherein said crude pentafluoroethane used in the step (2) is obtained through a further step of being contacted with hydrogen.
